(19) United States
(12) Patent Application Publication
(10) Pub. No.: US 2005/0053941 A1
(43) Pub. Date: Mar. 10, 2005

(54) Extraction of nucleic acid

(76) Inventors: Matthew Baker, Maidstone (GB); Matthew Taylor, Carterbury (GB); Shilpa Uppal, Canterbury (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/496,449

(22) PCT Filed: Nov. 20, 2002
(86) PCT No.: PCT/GB02/05209

(30) Foreign Application Priority Data
Nov. 20, 2001 (GB) 0127809.2

(51) Int. Cl. C12Q 1/68; C12N 1/08

(52) U.S. Cl. 435/6; 435/270

(57) ABSTRACT

Methods of obtaining a sample of target nucleic acid from cells containing the target nucleic acid and genomic DNA or RNA are disclosed. In contrast to prior art protocols, this method does not require the cells containing the target nucleic acid to be lysed and instead is based on the observation when cells are suspended in an aqueous medium and the target nucleic acid are released into the medium through the cell walls. The invention therefore helps to avoid the use of cell lysis, heating, extremes of pH, water immiscible solvents, and electrical fields used in prior art nucleic acid extraction methods. The present invention is particularly applicable to the separation of non-genomic nucleic acid, such as cellular vector DNA or RNA, self-replicating satellite nucleic acids or plasmid DNA, from genomic nucleic acids, such as host cell chromosomes and ribosomal RNA.

### FIELD OF THE INVENTION

The present invention relates to nucleic acid purification, and in particular to a method of obtaining a sample of target nucleic acid from cells containing the target nucleic acid and genomic DNA or RNA.

### BACKGROUND OF THE INVENTION

Current methods of purifying non-genomic nucleic acids, such as vector DNA or RNA, rely on extensive cell lysis or cell wall degradation to release the nucleic acid present in the cells, using cell lysing enzymes such as lysozyme, chaotropic agents or extremes of pH and heat (see for example the methods disclosed in Maniatis, ‘Molecular Cloning, A Laboratory Manual’, Book 1, Sections 1.21-1.23, Cold Spring Harbor Press, 1989). However, while this approach releases the non-genomic nucleic acid from the cells, it is accompanied by the majority of the other host genomic nucleic acid contained in the cell, and other impurities such as cellular endotoxins. This necessitates a sequence of difficult and time consuming purification steps in order purify target nucleic acid molecules from these impurities. These steps are needed as the release of RNA or host chromosomal DNA can then interfere with the downstream analysis or functionality of the target nucleic acid which, if present in the sample of the target nucleic acid. Further, extensive cell lysis often results in the generation of a viscous mass of cellular material making further purification difficult.

Calvin and Hanawalt (J. Bacteriol, 170(6): 2796-2801, 1988) discloses the use of reverse electroporation for the recovery of plasmid DNA from E. coli. While plasmids were recovered in the method, most of the conditions tested also led to the extraction of cellular DNA and RNA. In order to obtain the plasmids, electrical field strengths of 13,000-15,000 V/cm were required.

WO 00/29563 (Cambridge Molecular Technologies Limited) discloses a method of separating plasmid from genomic DNA employing a water immiscible organic solvent, a chaotrope and heating to at least 65° C. This led to the extraction of the plasmid DNA into the organic phase.

EP 0 657 530 A (Gen-Probe Inc) discloses a method of extracting nucleic acid from bacterial cells employing a permeabilising reagent comprising a non-ionic detergent and a metal chelating agent, such as EDTA, in combination with heating to 80-100° C.

An additional problem is that the procedures to purify a sample of the target nucleic acid from the impurities typically requires several centrifugation steps and during the purification procedures, the volume of the sample changes significantly. These factors mean that existing purification protocols need to be carried out by a technician and are not easily automated.

### SUMMARY OF THE INVENTION

Broadly, the present invention provides method of obtaining a sample of target nucleic acid from cells containing the target nucleic acid and genomic DNA or RNA. In contrast to prior art protocols, this method does not require the cells containing the target nucleic acid to be lysed and instead relies on the observation that after separation from culture broth or medium, cells can be resuspended in an aqueous medium and the target nucleic acid released into the aqueous medium through the cell walls without the need for cell lysis. The present invention may in preferred embodiments avoid the heating used in many prior art nucleic acid extraction methods. The present invention is particularly applicable to the separation of non-genomic nucleic acid, such as cellular vector DNA or RNA, self-replicating satellite nucleic acids or plasmid DNA, from genomic nucleic acids, such as host cell chromosomes and ribosomal RNA.

Accordingly, in a first aspect, the present invention provides a method of obtaining a sample of target nucleic acid from cells containing the target nucleic acid and genomic nucleic acid, the method comprising the steps of:
separating the cells from culture broth;
suspending the cells in an aqueous medium which causes the target nucleic acid to leak from the cells into the aqueous medium; and
obtaining the sample of the nucleic acid from the aqueous medium;
wherein the cells are substantially not lysed during the above steps and substantially retain the genomic nucleic acid within the cells

Preferably, the method does not substantially cause the release of cellular endotoxins, thereby allowing the separation of the target nucleic acid from the cellular endotoxins, in addition to genomic nucleic acid or RNA.

While not wishing to be bound by any particular theory, the inventors believe that the change in the environment of the cells from the conditions in culture broth to the conditions in the aqueous medium, typically at lower ionic strength, makes the cell walls ‘leaky’ or slightly porous to the target nucleic acid, and especially nucleic acid such as vectors. This allows the target nucleic acid to diffuse into the aqueous medium without the need to lyse the cells, e.g. by contacting the cells with lysozyme, chaotropes or extremes of pH or heat, with the result that the cells retain impurities such as genomic DNA and cellular proteins, thereby avoiding the need for complicated procedures to remove these materials from a sample comprising the impurities and the target nucleic acid. In a preferred embodiment of the invention, the target nucleic acids may be 100 kb or less, or more preferably 50 kb or less, or more preferably 20 kb or less or even more preferably 10 kb or less in size. The size of nucleic acids can be determined by those skilled in the art, e.g. using gel electrophoresis technique employing a polyacrylamide or agarose gel, e.g. see Ausubel et al, ‘Short Protocols in Molecular Biology’, John Wiley and Sons, NY, 1992.

In the present invention, “not substantially lysed” means preferably less than 20%, more preferably less than 10%, more preferably less than 5%, more preferably less than 2% and most preferably less than 1% of the cells in the population treated according to the method are lysed. The extent of cell lysis can readily be determined, e.g. by counting lysed and non-lysed cells present in a sample under a microscope.

Preferably, the target nucleic acid is non-genomic nucleic acid which is separated from genomic nucleic acid retained inside the cells. Non-genomic nucleic acid includes vectors, plasmids, self replicating satellite nucleic acid or cosmid DNA, or vector RNA. Other forms of target nucleic acids may include bacteriophages such as Lambda, M13 and viral nucleic acids. In a preferred embodiment, the non-genomic nucleic acid sample is plasmid DNA.

The method is widely applicable to many different types of host cells. For example Gram negative and Gram positive bacteria, filamentous bacteria or fungi such as Streptomyces, yeast cells, plant cells and plant protoplasts. The use of gram negative bacteria is preferred, such as E. Coli strains, e.g. strains JM109 or HB101.

Typically, the cells are treated at ambient temperatures between 0 and 90° C. Unlike many of the prior art methods, preferred embodiments of the present invention may employ temperatures less than about 60° C., and more preferably less than about 40° C. Preferred temperature ranges employed in the method of the invention are between 0 and 60° C., between 0 and 40° C. and most preferably between 15 and 40° C.

The present invention includes the use of a variety of different conditions to cause the release of target nucleic acid from the cells into the surrounding aqueous medium. Examples of preferred aqueous media include water, salts such as NaCl, in hypotonic or hypertonic conditions. Examples of low salt buffers include Tris. HCl, e.g. at pH 6-9, optionally including EDTA, a potassium salt such as potassium acetate, optionally including KCl, or a divalent or trivalent metal salt such as CaCl₂, preferably at a concentration between 10 and 250 mM, and more preferably between 50 and 150 mM.

Alternatively or additionally, a sugar solution can also be employed, preferably at concentrations between 0.05 and 1.0M, and more preferably between 0.1 and 0.5M. A preferred sugar solution is a sucrose solution.

Optionally, the aqueous media may additionally include a proteinase such as Proteinase K to improve the yield of the target nucleic acid. In particular, the inventors have found that including a proteinase with the divalent or trivalent metal ion salt greatly increases the leakage of the target nucleic acid from the cells. The use of Proteinase K and CaCl₂ is particularly preferred.

In a preferred embodiment, the aqueous medium is a low salt buffer. Preferably, the low salt buffer comprises Tris HCl, optionally in combination with EDTA. In this case, preferably the low salt buffer comprises 5 mM to 50 mM Tris HCl and most preferably about 10 mM Tris HCl. Where EDTA is present, it is preferably present at a concentration between 0.1 mM and 100 mM, more preferably at a concentration between 0.1 and 50 mM, and most preferably at a concentration of about 1 mM.

In another preferred embodiment, the low salt buffer is a potassium salt at a concentration between 10 mM and 30 mM, most preferably at a concentration of about 16 mM. A preferred pH of potassium salt solutions is about pH 4.

Preferably, the aqueous medium has a pH between pH 3 and 11, more preferably between pH 6 and 9, and most preferably a pH of about 8.5.

Slightly higher salt concentrations can also be used, e.g. by employing aqueous media including sodium chloride, e.g. at a concentration which is preferably between about 50 mM and 250 mM, and more preferably about 150 mM. A preferred pH of sodium chloride solutions is about pH 7. The salts solutions or aqueous media may be buffered with standard laboratory buffers such as biological buffers, e.g. MES, MOPS, HEPES or Acetate buffers or even phosphate buffers such as PBS.

The aqueous medium may comprise a detergent, either alone or in combination with one or more of the other components described herein. It is preferred that the detergent is a non-ionic detergent such as Tween 20 that do not inhibit subsequent use of the target nucleic acid, e.g. in analytical techniques. The aqueous medium may further comprise a RNA nuclease or a DNA nuclease to selectively degrade unwanted RNA or DNA targets from a mixture released into the surrounding medium. Additionally, a protease may be employed to degrade any released proteins.

The present invention makes it possible to avoid the harsh conditions used in many prior art methods for purifying nucleic acid. When conditions are made harsher, the cell wall degrades extensively and a considerable amount of unwanted nucleic acids are released. Examples of harsh conditions include, but are not limited to, 0.1M NaOH or 0.1M NaOH with 1% SDS. In some cases, harsh conditions include the use of temperatures of 65° C. or above and/or the use of water immiscible organic solvents and/or the use of electroporation. Thus, the method of the invention is preferably carried out in the absence of an electrical field capable of causing poration, e.g. having a field strength above 10,000 V/cm.

In some embodiments, the method may involve one or more of the additional steps of:
(a) isolating the target nucleic acid; or
(b) analysing the target nucleic acid; or
(c) amplifying the target nucleic acid; or
(d) sequencing the target nucleic acid.

These steps are discussed in more detail below.

In a further embodiment of the invention, the target nucleic acid, such as a plasmid, can be separated from the cells according to the present invention and the resulting aqueous media, i.e. the supernatant, used directly with out the requirement for further purification steps, e.g. for PCR or other analytical methods.

A range of techniques are available to the skilled person for purifying nucleic acid that has leaked from cells and is present in the aqueous media. Examples of purification techniques include ion-exchange, electrophoresis, silica solid phase with chaotropic salt extraction, precipitation, flocculation, filtration, gel filtration, centrifugation, alcohol precipitation and/or the use of a charge switch material described in our copending applications European Patent Application No: 98957019.7, U.S. patent application Ser. No. 09/736,632 and WO 02/48164 and other purification or separation methods well known in the art. In preferred embodiments, the target nucleic acid is purified using a charge switch material, e.g. present on a solid phase, a pipette tip, beads (especially magnetic beads), a porous membrane, a frit, a sinter, a probe or dipstick, a tube (PCR tube, Eppendorf tube) or a microarray. Charge switch materials may be solid phases or soluble. They comprise ionisable groups such that they are capable of binding nucleic acid at a first pH (typically between pH 5.0 and 9.0), and then releasing it at a second higher pH (typically less than 10.5). Examples of solid phase charge switch materials are those in which (1) the ionisable groups are separately immobilised on a solid support by covalent or ionic bonding or by adsorption, (2) the ionisable groups are separately attached to a polymer, said polymer being immobilised on a solid support by covalent or ionic bonding or by adsorption, (3) the ionisable groups are polymerised, optionally by means of cross-linking reagents, and the polymer is immobilised on a solid support by covalent or ionic bonding or by adsorption. By way of example, the charged groups in the charge switch material may be provided by a biological buffer (e.g. Tris, Bis-Tris, polyTris or poly Bis-Tris), a polyhydroxylated amine, a detergent or surfactant, a carbohydrate, a nucleic acid base, a heterocyclic nitrogen-containing compound, a monoamine, a biological dye, or a negatively ionisable group, the pKa of which is between about 3.0 and 7.0 and a metal oxide which is positively charged at said first pH, and optionally also at said second pH.

The target nucleic acid may also be the subject of amplification, conveniently using the polymerase chain reaction. PCR techniques for the amplification of nucleic acid are described in U.S. Pat. No. 4,683,195. In general, such techniques require that sequence information from the ends of the target sequence is known to allow suitable forward and reverse oligonucleotide primers to be designed to be identical or similar to the polynucleotlde sequence that is the target for the amplification. PCR comprises steps of denaturation of template nucleic acid (if double-stranded), annealing of primer to target, and polymerisation. The nucleic acid probed or used as template in the amplification reaction may be genomic DNA, cDNA or RNA. PCR can be used to amplify specific sequences from genomic DNA, specific RNA sequences and cDNA transcribed from mRNA, bacteriophage or plasmid sequences. References for the general use of PCR techniques include Mullis et al, Cold Spring Harbor Symp. Quant. Biol., 51:263, (1987), Ehrlich (ed), PCR Technology, Stockton Press, NY, 1989, Ehrlich et al, Science, 252:1643-1650, (1991), “PCR protocols; A Guide to Methods and Applications”, Eds. Innis et al, Academic Press, New York, (1990).

In some embodiments, the method may involve one or more initial steps such as:
culturing the cells in the culture broth or growing on selective media on plates. The cells/colonies are separated and then placed in the aqueous medium. Separation may be achieved by centrifugation, filtration, magnetic bead separation, or using a probes or dipsticks.

Thus, in one embodiment, the method comprises growing the cells in broth or culture medium, separating the cells and resuspending them in the aqueous media to cause the cells to become leaky and release the target nucleic acid.

Preferred embodiments of the present invention will now be described in more detail, by way of example and not limitation, with reference to the accompanying figures.

### BRIEF DESCRIPTION OF THE FIGURES

FIGS. 1 to 5 show the plasmid DNA samples purified according to the present invention run on agarose gels stained with ethidium bromide. The figures are labelled with the conditions (reagents and concentrations) used for each purification. The nucleic acid bands are all from plasmid DNA, in supercoiled, nicked or linear forms. No bands were observed for genomic DNA, demonstrating the selectivity of the method of the invention.

### EXAMPLE 1

An overnight 5 ml culture of JM109 containing pUC19 plasmid was centrifuged to pellet the cells which were then resuspended in 500 μl of 10 mM Tris. HCl, 10 mM EDTA pH 8.5. Following a 5 minute incubation, the cells were centrifuged leaving a clear supernatant containing the plasmid. A sample was taken for PCR analysis and the remaining plasmid was purified by adjusting the pH to 4 with 166 μl of a 1.6M potassium acetate buffer and then adding 1 mg of magnetic beads derivatised with positively charged groups. The magnetic beads were incubated for 1 minute to bind the plasmid, separated on a magnet, washed twice with water and the DNA recovered by eluting with 100 μl of 10 mM Tris.HCl pH 8.5. Plasmid purity was confirmed by gel electrophoresis and identity confirmed by PCR and sequencing.

### EXAMPLE 2

An overnight 5 ml culture of XL-1 Blue containing pUC19 plasmid was centrifuged to pellet the cells which were then resuspended in 500 μl of 10 mM Tris. HCl, 10 mM EDTA pH 8.5. Following a 5 minute incubation, the cells were centrifuged leaving a clear supernatant containing the plasmid. A sample was taken for PCR analysis and the remaining plasmid was purified by adjusting the pH to 4 with 166 μl of a 1.6M potassium acetate buffer and then adding 1 mg of magnetic beads derivatised with positively charged groups. The magnetic beads were incubated for 1 minute to bind the plasmid, separated on a magnet, washed twice with water and the DNA recovered by eluting with 100 ul of 10 mM Tris.HCl pH 8.5. Plasmid purity was confirmed by gel electrophoresis and identity confirmed by PCR and sequencing.

### EXAMPLE 3

An overnight 5 ml culture of XL-1 Blue containing pUC19 plasmid was centrifuged to pellet the cells which were then resuspended in 500 μl of water. Following a 5 minute incubation, the cells were centrifuged leaving a clear supernatant containing the plasmid. Plasmid purity was confirmed by gel electrophoresis and identity confirmed by PCR.

### EXAMPLE 4

An overnight 5 ml culture of XL-1 Blue containing pUC19 plasmid was centrifuged to pellet the cells which were then resuspended in 500 μl of 0.15M NaCl. Following a 5 minute incubation, the cells were centrifuged leaving a clear supernatant containing the plasmid. Plasmid purity was confirmed by gel electrophoresis and identity confirmed by PCR.

### EXAMPLE 5

An overnight 5 ml culture of XL-1 Blue containing pUC19 plasmid was centrifuged to pellet the cells which were then resuspended in 500 μl of 10 mM potassium acetate, 10 mM potassium chloride pH 4. Following a 5 minute incubation, the cells were centrifuged leaving a clear supernatant containing the plasmid. Plasmid purity was confirmed by gel electrophoresis and identity confirmed by PCR.

### EXAMPLE 6

An overnight 5 ml culture of XL-1 Blue containing pUC19 plasmid was centrifuged to pellet the cells which were then resuspended in 500 μl of 10 mM Tris HCl, 1% Tween 20. Following a 5 minute incubation, the cells were centrifuged leaving a clear supernatant containing the plasmid. Plasmid purity was confirmed by gel electrophoresis and identity confirmed by PCR.

### EXAMPLE 7

An overnight 5 ml culture of XL-1 Blue containing pUC19 plasmid was centrifuged to pellet the cells which were then resuspended in 500 μl of 10 mM Tris HCl, 1% Tween 20. Following a 5 minute incubation, the cells and supernatant containing the plasmid were added to a PCR reaction for direct analysis.

### EXAMPLE 8

An overnight 5 ml culture of JM109 containing pUC19 plasmid was centrifuged to pellet the cells which were then resuspended in 500 μl of 10 mM Tris. HCl, 10 mM EDTA pH 8.5 plus RNase A at 20 ug/ml. Following a 5 minute incubation, the cells were centrifuged leaving a clear supernatant containing the plasmid. A sample was taken for PCR analysis and the remaining plasmid was purified by adjusting the pH to 4 with 166 μl of a 1.6M potassium acetate buffer and then adding 1 mg of magnetic beads derivatised with positively charged groups. The magnetic beads were incubated for 1 minute to bind the plasmid, separated on a magnet, washed twice with water and the DNA recovered by eluting with 100 μl of 10 mM Tris.HCl pH 8.5. Plasmid purity was confirmed by gel electrophoresis

### EXAMPLE 9

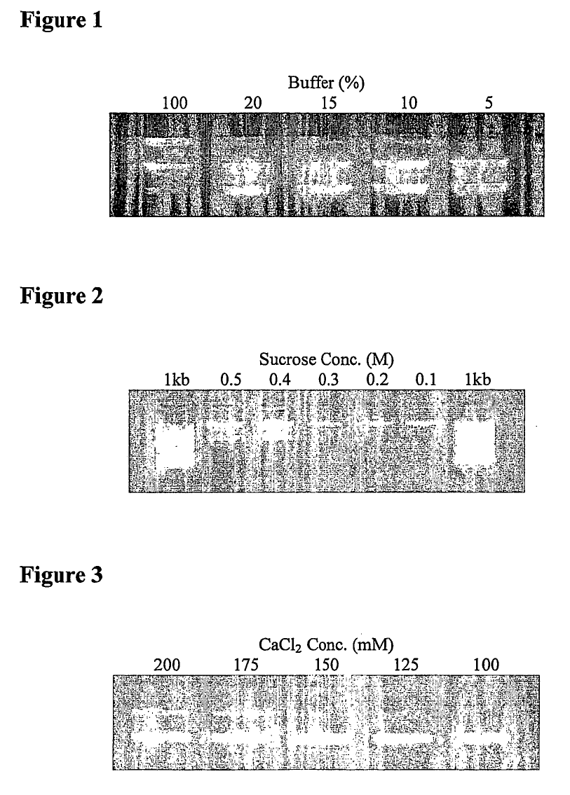

A solution of 10 mM Tris HCl, 10 mM EDTA, pH 8.5 was diluted with water to the following percentage concentrations (%): 100, 20, 15, 10 and 5. Then, 5×1.5 ml cultures from 200 ml pUC19/XL1-Blue overnight culture were spun down to pellet the cells. The supernatant was removed. About 500 μls of the range of buffer concentrations were added to the 5 tubes with 5 μl Proteinase K (20 mg/ml) and 5 μl RNase A (5 mg/ml). The 5 samples were fully resuspended and incubated at room temperature for 15 mins. After 15 mins, the samples were spun down again to pellet the cells. The supernatant was collected to a new tube and the pellet was discarded. To each tube, 500 μl of a 1.5 M potassium acetate buffer pH 4 (PB) with 40 μl of charge switch magnetic beads were added and the tubes were fully resuspended, incubated for 1 min, separated on a magnetic rack, washed twice and eluted in 50 μl Elution Buffer (EB). 10 μl of each sample was run on a 1% agarose gel stained with ethidium bromide as shown in FIG. 1.

### EXAMPLE 10

Sucrose was made up to the following concentrations (M): 0.5, 0.4, 0.3, 0.2 and 0.1. 5×1.5 ml cultures from a 200 ml pUC19/XL1-Blue overnight culture were spun down to pellet the cells. The supernatant was removed. 500 μls of the range of sucrose concentrations (M) were added to the 5 tubes. 5 μl Proteinase K and 5 μl RNase A was added to each tube as before. The 5 samples were fully resuspended and incubated at room temperature for 15 mins. After 15 mins, the samples were spun down again to pellet the cells. The supernatant was collected to a new tube and the pellet was discarded. 500 μl of PB and 40 μl of CST magnetic beads were added and the tubes were fully resuspended, incubated for 1 min, separated on a magnetic rack, washed twice and eluted in 50 μl Elution Buffer (EB). 10 μl of each sample was run on a 1% agarose gel stained with ethidium bromide, as shown in FIG. 2.

### EXAMPLE 11

CaCl₂ was made up to the following concentrations (mM): 200, 175, 150, 125 and 100. Five×1.5 ml cultures from a 200 μl pUC19/XL1-Blue overnight culture were spun down to pellet the cells. The supernatant was removed. About 500 μls of the range of CaCl₂ concentrations were added to the 5 tubes. 5 μl Proteinase K and 5 μl RNase A was added to each tube as before. The 5 samples were fully resuspended and incubated at room temperature for 15 mins. After 15 mins, the samples were spun down again to pellet the cells. The supernatant was collected to a new tube and the pellet was discarded. 500 μl of PB and 40 μl of charge switch magnetic beads were added and the tubes were fully resuspended, incubated for 1 min, separated on a magnetic rack, washed twice and eluted in 50 μl Elution Buffer (EB). 10 μl of each sample was run on a 1% agarose gel stained with ethidium bromide, as show in FIG. 3.

### EXAMPLE 12

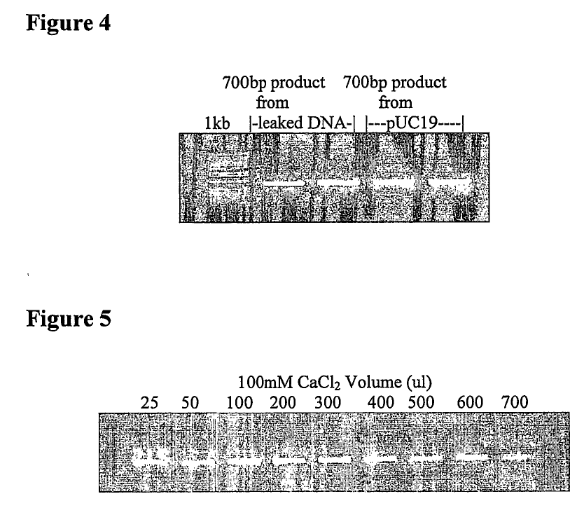

To confirm that the leaked DNA is pUC19 vector plasmid, 4 PCR reactions were set up using pUC19 vector primers to amplify a 700 bp PCR fragment. 2 were set up using DNA from a leaky cell extraction and 2 were set up using a sample of pUC19 from laboratory vector stocks. 10 μls of each product was run on an ethidium bromide stained 1% agarose gel with a 1 kb extension ladder, see FIG. 4.

### EXAMPLE 13

Nine×1.5 ml cultures from a 200 μl pUC19/XL1-Blue overnight culture were spun down to pellet the cells. The supernatant was removed. The following range of 100 mM CaCl₂ volumes (μl) were added to the 9 samples: 25, 50, 100, 200, 300, 400, 500, 600 and 700. 5 μl Proteinase K and 5 μl RNase A was added to each tube as before. The 9 samples were fully resuspended and incubated at room temperature for 15 mins. After 15 mins, the samples were spun down again to pellet the cells. The supernatant was collected to a new tube and the pellet was discarded. 500 μl of precipitation buffer and 40 μl of CST magnetic beads were added and the tubes were fully resuspended, incubated for 1 min, separated on a magnetic rack, washed twice and eluted in 50 μl Elution Buffer (EB). 10 μl of each sample was run on a 1% agarose gel stained with ethidium bromide, see FIG. 5.

The references cited herein are all expressly incorporated by reference in their entirety.

1. A method of obtaining a sample of target nucleic acid from cells containing the target nucleic acid and genomic nucleic acid, the method comprising the steps of:
separating the cells from culture broth;
suspending the cells in an aqueous medium which causes the target nucleic acid to leak from the cells into the aqueous medium; and
obtaining the sample of the nucleic acid from the aqueous medium;
wherein the cells are substantially not lysed during the above steps and substantially retain the genomic nucleic acid within the cells.

2. The method of claim 1, wherein the method is carried out at a temperature of less than 60° C. and in the absence of an electrical field capable of causing cell poration.

3. The method of claim 2, wherein the method is carried out a temperature of less than 40° C.

4. The method of claim 1, wherein cellular proteins are substantially retained within the cells.

5. The method of claim 1, wherein the target nucleic acid is non-genomic nucleic acid.

6. The method of claim 5, wherein the genomic nucleic acid is host cell chromosomal DNA or ribosomal RNA.

7. The method of claim 1, wherein the target nucleic acid sample is a vector, a plasmid, satellite or cosmid DNA or vector RNA.

8. The method of claim 7, wherein the target nucleic acid sample is plasmid DNA.

9. The method of claim 1, wherein the cells are a gram negative microorganism.

10. The method of claim 9, wherein the cells are E. coli.

11. The method of claim 1, wherein the aqueous medium is water, a low salt buffer, a salt solution, or a sugar solution.

12. The method of claim 1, wherein the aqueous medium has a pH between pH 6 and 9.

13. The method of claim 11, wherein the aqueous medium is a low salt buffer comprising Tris. HCl.

14. The method of claim 13, wherein the Tris. HCl buffer has a concentration between 5 mM and 50 mM.

15. The method of claim 13, wherein the Tris. HCl buffer further comprises EDTA.

16. The method of claim 13, wherein the Tris. HCl buffer has a pH of about 8.5.

17. The method of claim 11, wherein the aqueous medium is a low salt buffer comprising potassium acetate/KCl.

18. The method of claim 17, wherein the potassium acetate/KCl has a concentration between 10 mM and 30 mM.

19. The method of claim 17, wherein the potassium acetate/KCl has a pH of about 4.

20. The method of claim 11, wherein the aqueous medium is a salt solution.

21. (Cancelled)

22. (Cancelled)

23. (Cancelled)

24. The method of claim 11, wherein the aqueous medium is a sugar solution.

25. The method of claim 1, wherein the aqueous medium further comprises a proteinase.

26. The method of claim 25, wherein the proteinase is Proteinase K.

27. The method of claim 1, wherein the aqueous medium further comprises a detergent.

28. The method of claim 27, wherein the detergent is a non-ionic detergent.

29. The method of claim 1, wherein the aqueous medium further comprises a RNA nuclease, a DNA nuclease a protease or a combination of two or more of said enzymes.

30. The method of claim 1, further comprising purifying the target nucleic acid present in the sample of target nucleic acid.

31. The method of claim 1, further comprising isolating the sample of the target nucleic acid.

32. The method of claim 1, further comprising analysing the sample of the target nucleic acid.

33. The method of claim 1, further comprising amplifying the sample of the target nucleic acid.

34. The method of claim 1, further comprising sequencing the sample of the target nucleic acid.

35. The method of claim 31, wherein the isolation of the target nucleic acid is by ion-exchange, electrophoresis, silica solid phase extraction, precipitation, flocculation, filtration, gel filtration, centrifugation, alcohol precipitation and the use of a charge switch material.

36. The method of claim 20, wherein said salt solution is a sodium chloride solution.

37. The method of claim 36, wherein the sodium chloride solution has a concentration between about 50 mM and 250 mM and/or a pH of about pH 7.

38. The method of claim 20, wherein said salt solution is a divalent or trivalent metal salt solution.

39. The method of claim 38, wherein the aqueous medium is a divalent metal ion solution.

40. The method of claim 39 wherein the divalent metal in solution is a CaCl₂ solution.

41. The method of claim 40, wherein the CaCl₂ solution is at a concentration between 10 and 250 mM.

42. The method of claim 24 wherein the sugar solution is a sucrose solution having a concentration between 0.05 and 1.0M.

43. The method of claim 31, wherein the isolation of the target nucleic acid is by ion-exchange, electrophoresis, silica solid phase extraction, precipitation, flocculation, filtration, gel filtration, centrifugation, alcohol precipitation or the use of a charge switch material.
